(19) United States
(12) Patent Application Publication
(10) Pub. No.: US 2006/0287716 A1
(43) Pub. Date: Dec. 21, 2006

(54) Artificial chordae

(75) Inventors: Michael K. Banbury, Rockland, DE (US); A. Marc Gillinov, Orange Village, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/448,629

(22) Filed: Jun. 7, 2006

Related U.S. Application Data
(60) Provisional application No. 60/688,455, filed on Jun. 8, 2005.

(51) Int. Cl. A61F 2/24

(52) U.S. Cl. 623/2.1; 623/2.16

(57) ABSTRACT

An apparatus for replacing the native chordae of a heart valve having at least two leaflets includes a prosthetic chordae assembly configured to extend from a papillary muscle to one of the at least two valve leaflets of the heart valve. The prosthetic chordae assembly has first and second end portions, and a middle portion extending therebetween. The prosthetic chordae assembly further includes a plurality of loop members interconnected at the first end portion for suturing to the papillary muscle. The middle portion is formed by two generally parallel strands of each of the loop members, and the second end portion is formed by an arcuate junction of the two strands of each of the loop members. The arcuate junctions are spaced apart and each of the junctions provides an independent location for attaching to one of the at least two valve leaflets of the heart valve.

### RELATED APPLICATION

This application claims priority from U.S. provisional patent application Ser. No. 60/688,455, filed Jun. 8, 2005, the subject matter of which is incorporated herein by reference.

### TECHNICAL FIELD

The present invention relates to artificial chordae, and more particularly to a prosthetic chordae assembly for a mitral or tricuspid valve.

### BACKGROUND OF THE INVENTION

Heart valve replacement is a well known procedure in which an artificial heart valve prosthesis is implanted in place of a diseased or malfunctioning heart valve. Heart valve prostheses may be mechanical or bioprosthetic. Use of mechanical valves typically requires extensive anticoagulation therapy. The need for anticoagulation therapy can be avoided in general by the use of artificial biological heart valves, such as bovine xenografts. Nevertheless, dystrophic calcification with subsequent degeneration is the major cause of failure of such bioprostheses in the long term.

When mitral or tricuspid valve replacement is performed, the chordae tendineae are cut, thus leaving the geometry and function of the ventricle impaired and in need of reconstruction. As an alternative to conventional heart valve replacement operations, diseased and malfunctioning chordae can be repaired by surgically replacing diseased heart chordae with artificial chordae. One known way of replacing a malfunctioning chordae uses a simple suture with a needle on each end of the suture. The suture is stitched through the papillary muscle and secured thereto with a knot. The two ends of the suture are then similarly stitched through the free ends of the valve leaflets.

Operations to repair heart valve chordae are technically demanding. For example, when a second knot is needed to secure the suture to the valve leaflets, the length of the suture spanning the distance between the papillary muscle and the valve leaflet is likely to change since there is nothing holding the suture in place. This complication increases the skill and time required to perform the procedure. Moreover, the valve will not function properly if the length of the artificial chordae between the papillary muscle and valve leaflet is overly long or overly short.

### SUMMARY OF THE INVENTION

In one aspect of the present invention, an apparatus for replacing the native chordae of a heart valve having at least two leaflets comprises a prosthetic chordae assembly configured to extend from a papillary muscle to one of the at least two valve leaflets of the heart valve. The prosthetic chordae assembly has a first end portion, a second end portion, and a middle portion extending between the end portions. The prosthetic chordae assembly further comprises a plurality of loop members interconnected at the first end portion for suturing to the papillary muscle. The middle portion is formed by two generally parallel strands of each of the loop members, and the second end portion is formed by an arcuate junction of the two strands of each of the loop members. The arcuate junctions are spaced apart and each of the junctions provides an independent location for attaching to one of the at least two valve leaflets of the heart valve.

In another aspect of the present invention, a method is provided for replacing the native chordae of a heart valve having at least two leaflets. First, the distance between a papillary muscle and a location on at least two leaflets of the heart is measured. Based on the measured distance, a prosthetic chordae assembly configured to extend from the papillary muscle to the location on the at least two leaflets of the heart valve is then selected. After selecting the appropriate prosthetic chordae assembly, each of the arcuate junctions is attached at a selected position to an independent, spaced apart location on one of the at least two leaflets of the heart valve, and the first end portion is attached to the papillary muscle.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a plan view of an apparatus for replacing the native chordae of a heart valve having at least two leaflets constructed in accordance with the present invention;

FIG. 2 is an alternative embodiment of the apparatus shown in FIG. 1;

FIG. 3 is a cross-sectional view of a human heart; and

FIG. 4 is a cross-sectional view showing the apparatus of FIG. 1 implanted in a human heart.

### DETAILED DESCRIPTION

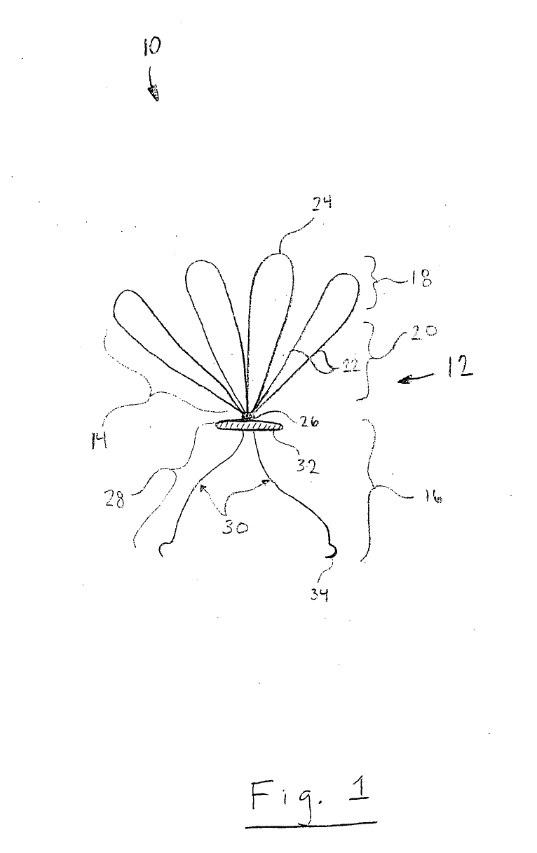

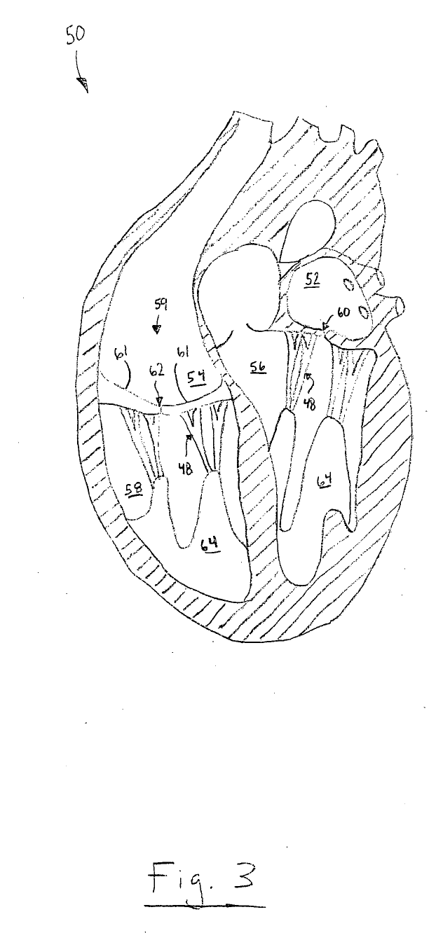

The present invention relates to artificial chordae, and more particularly to a prosthetic chordae assembly for a mitral or tricuspid valve. As representative of the present invention, FIG. 1 illustrates an apparatus 10 for replacing the native chordae 48 of a heart valve 59 having at least two leaflets 61 (FIG. 3). The apparatus 10 of the present invention comprises a prosthetic chordae assembly 12 (FIG. 1) configured to extend from a papillary muscle 64 (FIG. 3) to one of the at least two valve leaflets 61 of the heart valve 59. The prosthetic chordae assembly 12 (FIG. 1) comprises a plurality of loop members 14, and has a first end portion 16, a second end portion 18, and a middle portion 20 extending between the end portions.

Each of the loop members 14 of the prosthetic chordae assembly 12 comprise two generally parallel strands 22 and an arcuate junction 24. The two generally parallel strands 22 are fluidly connected to the arcuate junction 24 of each loop member 14. Each loop member 14 has a size and length that is equal to the size and length of other loop members. Alternatively, each loop member 14 may have a length and size different from each of the other loop members. Each loop member 14 is made from a biocompatible material that is relatively inelastic and flexible to allow easy movement of the heart valve leaflets 61 during opening and closing of the valve 59. Examples of suitable biocompatible materials include Teflon and expanded polytetrafluoroethylene (ePTFE). The ePTFE may be suture material or fabric material. Besides Teflon and ePTFE, it should be apparent to one skilled in the art that there are other suitable biocompatible materials, including those which are frequently used to form sutures.

The prosthetic chordae assembly 12 may comprise a unitary unit. For example, the prosthetic chordae assembly 12 may be comprised of a single strand or fiber, such as a suture. Alternatively, the prosthetic chordae assembly 12 may be comprised of multiple units, e.g., multiple sutures. Where the prosthetic chordae assembly 12 is comprised of multiple sutures, each of the sutures is fixedly joined to form the loop members 14.

The first end portion 16 of the prosthetic chordae assembly 12 comprises a plurality of interconnected loop members 14. Each loop member 14 may be interconnected at a common junction 26. Where the prosthetic chordae assembly 12 comprises a unitary unit, the common junction 26 may be formed by grouping each loop member 14 and then tying a knot to secure each loop member at the common junction. The knot may be formed by tying a portion of the prosthetic chordae assembly 12 (e.g., using an end of the suture) around the common junction 26. Alternatively, the knot may be tied by using a separate material, e.g., a separate suture. Other methods may also be used to secure each loop member 14 at the common junction 26. For instance, each loop member 14 may be secured at the common junction 26 by gluing, stapling, pinning, or any other suitable method.

The common junction 26 may also be formed where the prosthetic chordae assembly 12 is comprised of multiple units. First, each loop member 14 may be separately formed. Each loop member 14 may then be grouped together and joined at the common junction 26 by tying a knot. Alternatively, each loop member 14 may be joined by suturing, gluing, pinning, stapling or any other suitable method.

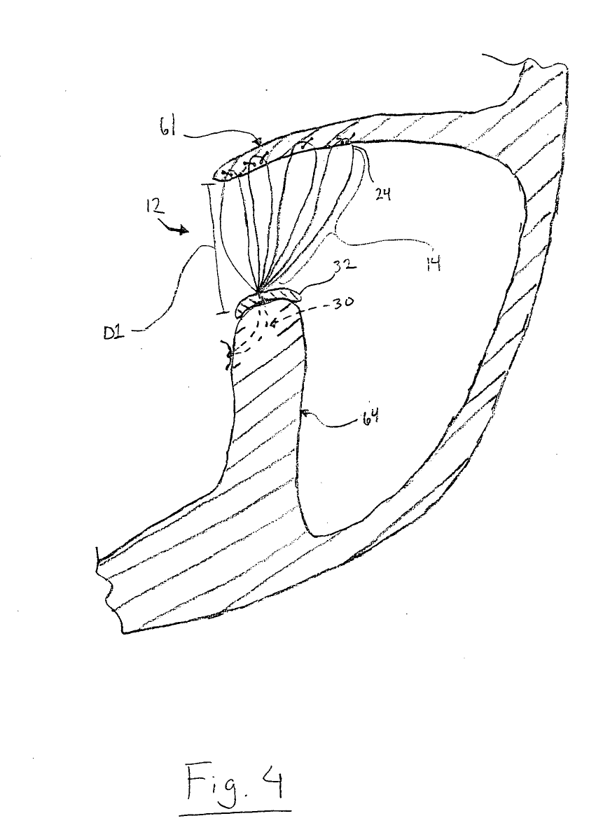

The first end portion 16 of the prosthetic chordae assembly 12 may further comprise an attachment mechanism 28 for securing the apparatus 10 to the papillary muscle 64. One embodiment of the attachment mechanism 28 is illustrated in FIGS. 1 and 4. As shown in FIG. 1, the attachment mechanism 28 is comprised of oppositely disposed strands 30 and at least one pledget 32. Each of the oppositely disposed strands 30 may include a needle 34 capable of penetrating the papillary muscle 64. Alternatively, the oppositely disposed strands 30 may include clamps, pins, fasteners, barbs, or any other means capable of penetrating or otherwise affixing the apparatus 10 to the papillary muscle 64. Where the prosthetic chordae 12 assembly is formed from a unitary unit, the oppositely disposed strands 30 comprise the ends of the unitary unit. Alternatively, where the prosthetic chordae assembly 12 is comprised of multiple units, the oppositely disposed strands 30 may be formed from separate strands, each strand being fixedly attached to the common junction 26.

The attachment mechanism 28 of the first end portion 16 may also include at least one pledget 32 fixedly attached to the first end portion of the apparatus 10. Alternatively, the pledget 32 may be slidably attached to the first end portion 16 to facilitate positing or suturing of the prosthetic chordae assembly 12. The pledget 32 may be comprised of a non-absorbable material such as polyurethane, silicon, polyvinyl acetate, neoprene, or Teflon foam. Alternatively, the pledget 32 may be comprised of an absorbable material such as gelatin or collagen.

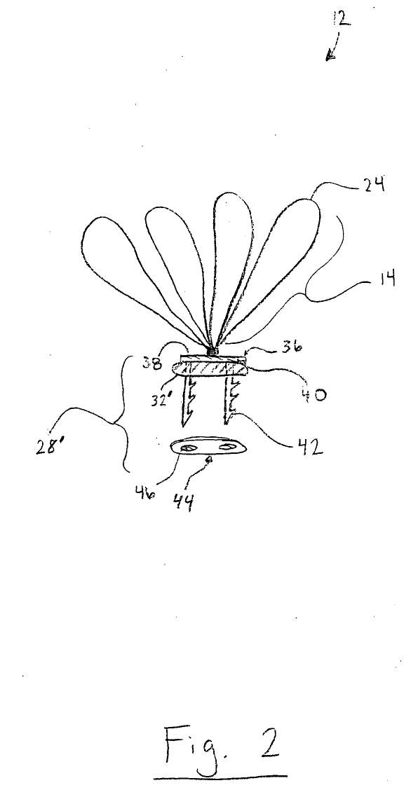

FIG. 2 shows an alternative embodiment of the attachment mechanism 28′. The attachment mechanism 28′ may comprise a substrate 36 having first and second sides 38 and 40, at least one barb member 42 attached thereto, a securing member 44, and at least one pledget 32. As illustrated in FIG. 2, the first end portion 16 of the prosthetic chordae assembly 12 comprises a plurality of loop members 14 fixedly attached to the first side 38 of the substrate 36. The loop members 14 may be attached to the first side 38 of the substrate 36 by, for example, gluing, suturing, stapling, or other similar method. The substrate 36 may be comprised of a biocompatible material that is flexible and resiliently yieldable. For instance, the substrate 36 may be made of ePTFE, silicon, Teflon, or other similar material.

The barb member 42 may be fixedly attached to the second side 40 of the substrate 36. As shown in FIG. 2, the barb member 42 can be needle-shaped. The skilled artisan will appreciate that the shape of the barb member 42 shown in FIG. 2 is illustrative, and is not intended to be exhaustive. Rather, it should be understood by the skilled artisan that the barb member 42 may have any shape that effectively allows the barb member to penetrate biological tissue. The barb member 42 may be made of a biologically compatible material such as hardened plastic, titanium, stainless steel, or other similar material.

The securing member 44 is comprised of a biocompatible material adapted to mate with the barb member 12. As shown in FIG. 2, the securing member 44 comprises a biocompatible material having two slots 46 particularly adapted to mate with the two barb members 42. The securing member 44 can be made from a biocompatible material that is flexible and resiliently yieldable. Examples of suitable materials include ePTFE, silicon, Teflon and other similar materials.

The attachment mechanism 28′ of FIG. 2 may also comprise at least one pledget 32′. The pledget 32′ may be fixedly attached to the second side 40 of the substrate 36. Alternatively, the pledget 32′ may be fixedly attached to the securing member 44 such that the pledget contacts the barb member 42 and the second side 40 of the substrate 36 when the securing member is mated with the barb member. The pledget 32′ may be comprised of a non-absorbable material such as polyurethane, silicon, polyvinyl acetate, neoprene, or Teflon foam. Alternatively, the pledget 32′ may be comprised of an absorbable material such as gelatin or collagen.

The middle portion 20 of the prosthetic chordae assembly 12 is formed from two generally parallel strands 22 of each of the loop members 14. The middle portion 20 extends to the second end portion 18 of the prosthetic chordae assembly 12. The second end portion 18 of the prosthetic chordae assembly 12 is formed by an arcuate junction 24 of the two generally parallel strands 22 of each of the loop members 14. The arcuate junctions 24 are spaced apart so that each of the arcuate junctions provides an independent location for attaching to one of the at least two valve leaflets 61 of the heart valve 59.

The replacement of native chordae 48 with the present invention is illustrated in FIGS. 3 and 4. A human heart 50 is schematically shown in FIG. 3. The heart 50 includes the left and right atria 52 and 54, and the left and right ventricles 56 and 58. The mitral valve 60 is between the left atrium 52 and the left ventricle 56, and the tricuspid valve 62 is similarly located between the right atrium 54 and right ventricle 58. In the mitral valve 60, the free edges of the mitral valve leaflets are connected to the papillary muscle 64 by the chordae tendineae 48. Similarly, the free edges of the tricuspid valve 62 are connected to the papillary muscle 64 by the chordae tendineae 48.

To replace native chordae 48, the present invention comprises a first step of measuring the distance D1 (FIG. 4) between a papillary muscle 64 and a location on at least one of the two leaflets 61 of a heart valve 59. Access to the chordae 48 may be obtained by open-heart surgery. During open-heart surgery, a physician inspects the native chordae 48 and determines the size of the prosthetic chordae assembly 12 needed to replace the native chordae. Generally, the size of the prosthetic chordae assembly 12 needed depends on the size of the heart 50 as well as the point of placement chosen by the physician. Various means for measuring the distance between the papillary muscle 64 and the heart valve 59 are known in the art. U.S. Patent App. No. 2003/0105519A1, for example, discloses a heart valve chordae sizing gauge for measuring the distance between the papillary muscle 64 and a valve leaflet 61.

After measuring the distance D1 between the papillary muscle 64 and the location on at least one of the two leaflets 61 of the heart valve 59, an appropriately sized prosthetic chordae assembly 12 is selected by the physician. The size of the loop members 14 define the length of the implanted prosthetic chordae assembly 12 in place in the patient's heart. Accordingly, a prosthetic chordae assembly 12 having a size equal to the measured distance D1 between the papillary muscle 64 and the location on at least one of the two leaflets 61 of the heart valve 59 is selected by the physician. After selecting the appropriately-sized prosthetic chordae assembly 12, the physician may excise the native chordae 48 and proceed to attach the apparatus 10. Alternatively, the physician may choose not to excise the native chordae 48 when attaching the apparatus 10. Failure to accurately determine the correct size of the loop members 14 may result in an ineffective repair, causing prolapse of the leaflet 61, which in turn may cause the heart valve 59 to leak.

As illustrated in FIG. 4, the prosthetic chordae assembly 12 may be secured to the papillary muscle 64 by passing the oppositely disposed strands 30 of the attachment mechanism 28 through the papillary muscle and then tying the oppositely disposed strands into a knot. Alternatively, the attachment mechanism 28′ shown in FIG. 2 may be used to secure the prosthetic chordae assembly 12 to the papillary muscle 64. Where the attachment mechanism 28′ shown in FIG. 2 is employed, the physician may first place the prosthetic chordae assembly 12 adjacent the papillary muscle 64 so that the barb member 42 contacts the papillary muscle. The physician may then apply pressure to the prosthetic chordae assembly 12 so that the barb member 42 penetrates, and then protrudes through, the papillary muscle 64. The securing member 44 may then be mated with the protruding barb member 42 so that the prosthetic chordae assembly 12 is fixedly attached to the papillary muscle 64. Such a device would facilitate the attachment and access to the remote location of the papillary muscle 64 during surgery.

During attachment of the prosthetic chordae assembly 12 to the papillary muscle 64, it is critical that the physician does not exert undue pressure on the papillary muscle. Excess pressure on the papillary muscle 64 may crush or deform the papillary muscle, in turn rendering the papillary muscle dysfunctional. Therefore, the attachment mechanism 28 and 28′ shown in FIGS. 1 and 2 may be applied using tactile feedback. For example, the physician may gauge the appropriate amount of pressure by using his or her hands. Alternatively, the physician may secure the attachment mechanism 28′ with assistance from a device (not shown) capable of preventing deformation or crushing of the papillary muscle 64. Further, it is contemplated that a specialized device (not shown) designed specifically for deploying and attaching the prosthetic chordae assembly 12 to the papillary muscle 64 could be utilized.

After securing the prosthetic chordae assembly 12 to the papillary muscle 64, the second end portion 18 of the prosthetic chordae assembly may be fixedly attached to the location on at least one of the leaflets 61 of the heart valve 59. The arcuate junction 24 of each loop member 14 can be positioned adjacent the location on the heart valve 50. Next, the physician can secure the arcuate junction 24 of each loop member 14 to the location on at least one of the leaflets 61 of the heart valve 59. For instance, the physician may secure the prosthetic chordae assembly 12 by placing a suture through both the arcuate junction 24 of each loop member 14 and the location on at least one of the leaflets 61 of the heart valve 50, and then tying the suture into a knot. The physician may also use other methods for attaching the loop members 14, such as with staples, clips, pins, biocompatible adhesives, constricting bands, or other suitable attachment devices. Alternatively, the physician may secure each of the arcuate junctions 24 to the location on at least one of the leaflets 61 of the heart valve 59 using a plurality of sutures. By using a plurality of sutures to secure each arcuate junction 24 to the location on at least one of the leaflets 61 of the heart valve 59, the tension at any one attachment point on the valve leaflet may be reduced. By reducing the tension at each attachment point, tearing or ripping of the valve leaflet 61 may be avoided.

It should be appreciated that the method of the present invention is not restricted to the order of steps presented herein. For example, the physician may first attach the prosthetic chordae assembly 12 to the location on at least one of the two leaflets 61 of the heart valve 59 instead of first attaching the prosthetic chordae assembly to the papillary muscle 64. As another example, the physician may attach the prosthetic chordae assembly 12 prior to excising the native chordae 48.

Upon successful placement of the prosthetic chordae assembly 12, the physician evaluates the patient's heart function and verifies that the valve leaflets 61 open and close effectively. After verifying effective heart function, the open-heart surgery is completed and the patient is provided with a normally functioning heart valve.

From the above description of the invention, those skilled in the art will perceive improvements, changes and modifications. Such improvements, changes and modifications within the skill of the art are intended to be covered by the appended claims.

Having described the invention, we claim:

1. Apparatus for replacing the native chordae of a heart valve having at least two leaflets, said apparatus comprising:
a prosthetic chordae assembly configured to extend from a papillary muscle to one of the at least two valve leaflets of the heart valve,
said prosthetic chordae assembly having a first end portion, a second end portion, and a middle portion extending between said end portions;
said prosthetic chordae assembly comprising a plurality of loop members interconnected at said first end portion for suturing to the papillary muscle;
said middle portion being formed by two generally parallel strands of each of said loop members;
said second end portion being formed by an arcuate junction of said two strands of each of said loop members, wherein said arcuate junctions are spaced apart and each of said junctions provides an independent location for attaching to one of the at least two valve leaflets of the heart valve.

2. A method for replacing the native chordae of a heart valve having at least two leaflets, said method comprising the steps of:
measuring the distance between a papillary muscle and a location on at least two leaflets of the heart valve;
selecting a prosthetic chordae configured to extend from the papillary muscle to the location on the at least two leaflets of the heart valve based on the measured distance, the prosthetic chordae assembly having a first end portion, a second end portion, and a middle portion extending between the end portions, the prosthetic chordae assembly comprising a plurality of loop members interconnected at the first end portion, the middle portion being formed by two generally parallel strands of each of the loop members, the second end portion being formed by an arcuate junction of the two strands of each of the loop members;
attaching each of the junctions at the selected position to an independent, spaced apart location on one of the at least two leaflets of the heart valve; and
attaching the first end portion to the papillary muscle.
